(19) United States
(12) Patent Application Publication
(10) Pub. No.: US 2008/0207772 A1
(43) Pub. Date: Aug. 28, 2008

(54) Mixture of Interference Pigments

(76) Inventors: Helge Bettina Kniess, Rossdorf (DE); Gerhard Pfaff, Munster (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/994,255

(22) PCT Filed: Jun. 9, 2006
(86) PCT No.: PCT/EP06/05566

§ 371 Date: Dec. 28, 2007

(30) Foreign Application Priority Data
Jun. 29, 2005 (DE) 10 2005 030 244.0

(51) Int. Cl. C09C 1/00; C09C 1/22; C09D 11/00; A61K 8/26; A61K 8/19; A61K 8/25; A61K 8/29; C09C 1/36

(52) U.S. Cl. 514/769; 106/459; 106/439; 106/418; 106/457; 106/31.65

(57) ABSTRACT

The present invention relates to a mixture of interference pigments, where the mixture comprises at least one interference pigment A comprising a substrate and a coating comprising metallic iron, and an interference pigment B comprising a substrate and a coating comprising metallic tin and in addition at least one metal oxide, to the use thereof in cosmetics, surface coatings, inks, plastics, films, in security printing, in security features in documents and identity papers, for colouring seed, for colouring foods, for laser marking or in medicament coatings and for the preparation of pigment compositions and dry preparations, and to cosmetics, surface coatings, inks, plastics, films, documents and identity papers, seed, foods or medicament coatings, pigment compositions and dry preparations comprising the mixture according to the invention.

The present invention relates to a mixture of interference pigments, where the mixture comprises at least one interference pigment A comprising a substrate and a coating comprising metallic iron, and an interference pig- ment B comprising a substrate and a coating comprising metallic tin and in addition at least one metal oxide, to the use thereof in cosmetics, surface coatings, inks, plastics, films, in security printing, in security features in documents and identity papers, for colouring seed, for colouring foods, for laser marking or in medicament coatings and for the preparation of pigment compositions and dry preparations, and to cosmetics, surface coatings, inks, plastics, films, documents and identity papers, seed, foods or medi- cament coatings, pigment compositions and dry preparations comprising the mixture according to the invention.

Mixtures of interference pigments are known to the person skilled in the art and are found, for example, in various surface-coating formulations in the area of automobile finishes.

Interference pigments, and thus also mixtures of these pigments, often do not exhibit the desired hiding power in surface-coating applications, in particular if they are based on semitransparent or transparent substrates, such as, for example, mica, SiO₂ or Al₂O₃. In order to ensure an adequate hiding power, carbon black is usually added to the pigment mixtures. However, the admixing of carbon black has the disadvantage that the carbon black can only be metered in with difficulty owing to its very strong hiding power. Only slight overdosing results in a very considerable increase in the hiding power, which in turn results, in order to achieve a desired colour impression, in intentional design incorporation of the substrate to which the surface coating is applied being made much more difficult, if not completely impossible. The establishment of a prespecified hiding power for the surface coating to be applied can thus only be controlled with difficulty.

The object was therefore to provide mixtures, in particular for surface-coating applications, which allow the establishment of a prespecified hiding power and at the same time exhibit attractive optical effects.

This object is achieved by mixtures in accordance with the present invention. The present invention accordingly relates to a mixture of interference pigments, where the mixture comprises at least one interference pigment A comprising a substrate and a coating comprising metallic iron, and an interference pigment B comprising a substrate and a coating comprising metallic tin and in addition at least one metal oxide.

Mixtures in accordance with the present invention are distinguished by the fact that the hiding power, for example of a surface coating, can be controlled well, i.e. a desired optical effect can be established more simply and accurately or can even only be achieved at all through inclusion of the substrate, for example through inclusion of the colour impression of the article or previously applied covering layers. In addition, it has been found, surprisingly, that the mixture in accordance with the present invention has particularly good angle dependence of the hiding power compared with mixtures comprising carbon black. Thus, on viewing of surfaces coated with the mixture according to the invention from flat angles, greater transparency is observed than on viewing from steeper angles. In particular in the case of three-coat finishes, as used, for example, in the automobile sector, this results in particularly attractive effects. Mixtures in accordance with the present invention are furthermore distinguished by the fact that they manage completely without carbon black or with very small additions of carbon black compared with conventional applications, in order to supplement the desired effects in the application system. Since the addition of carbon black is always associated with a loss of gloss of the interference pigment, high-gloss colour effects which simultaneously have high hiding power can be achieved with the mixtures according to the invention.

Owing to the above-mentioned advantages, the pigment mixtures according to the invention can be employed in a variety of areas. The present invention accordingly also relates to the use of the pigment mixtures in cosmetics, surface coatings, inks, plastics, films, in security printing, in security features in documents and identity papers, for colouring seed, for colouring foods, for laser marking or in medicament coatings and for the preparation of pigment compositions and dry preparations, and to cosmetics, surface coatings, inks, plastics, films, documents and identity papers, seed, foods or medicament coatings, pigment compositions and dry preparations comprising pigment mixtures in accordance with the present invention.

In the pigment mixture according to the invention, interference pigments comprising a substrate and a coating comprising metallic iron and interference pigments comprising a substrate and a coating comprising metallic tin and in addition at least one metal oxide are combined with one another in order to achieve the above-mentioned effects. The ratio of interference pigment A to interference pigment B can be matched to the particular requirements and is usually 99:1 to 1:99. The ratio of interference pigment A to interference pigment B is preferably 10:1 to 0.5:1. These preferred mixtures are particularly advantageous since, depending on the applicational properties to be achieved, they represent an optimum of gloss, hiding power and angle dependence of the hiding power.

In principle, the interference pigments A and B can have any conceivable shape, but they are preferably in flake form. The size of the interference pigments is not crucial per se. Flake-form interference pigments A and B generally have a thickness between 0.05 and 5 μm, in particular between 0.1 and 4.5 μm. The size of the interference pigments in the length and width can be between 1 and 250 μm, preferably in the range from 2 to 200 μm and very particularly preferably in the range from 2 to 100 μm. The size of the substrates can be matched to the requirements of the particular applications.

The interference pigments A are interference pigments comprising a substrate and a coating comprising metallic iron. The interference pigments B employed in the mixture comprise a substrate and a coating comprising metallic tin and in addition at least one metal oxide.

The substrate of the interference pigments A and B is independent of one another and can comprise synthetic or natural mica, phyllosilicates, glass, borosilicates, SiO₂, Al₂O₃, TiO₂, graphite and/or BiOCl. This means that the interference pigments A and B can be based on different substrates.

In a further embodiment of the present invention, one or more layers comprising metal oxides, metal oxide hydrates, metal suboxides, metals, metal fluorides, metal nitrides, metal oxynitrides and/or mixtures thereof may be present between the coating comprising metallic iron and/or between the coating comprising metallic tin and in addition at least one metal oxide and the substrate in the interference pigments A and B employed in the mixture according to the invention.

The metal oxide, metal oxide hydrate, metal suboxide, metal, metal fluoride, metal nitride or metal oxynitride layers or the mixtures thereof can be of low refractive index (refractive index<1.8) or high refractive index (refractive index≧1.8). Suitable metal oxides and metal oxide hydrates are all metal oxides or metal oxide hydrates to be applied as layers, such as, for example, aluminium oxide, aluminium oxide hydrate, iron oxide, tin oxide, cerium oxide, zinc oxide, zirconium oxide, chromium oxide, titanium oxide, in particular titanium dioxide, titanium oxide hydrate, and mixtures thereof, such as, for example, ilmenite or pseudobrookite. Metal suboxides which can be employed are, for example, the titanium suboxides. Suitable metals are, for example, iron, chromium, aluminium, nickel, silver, gold, titanium, copper or alloys, and a suitable metal fluoride is, for example, magnesium fluoride. Metal nitrides or metal oxynitrides which can be employed are, for example, the nitrides or oxynitrides of the metals titanium, zirconium and/or tantalum. Metal oxide, metal, metal fluoride and/or metal oxide hydrate layers and very particularly preferably metal oxide and/or metal oxide hydrate layers are preferably applied to the substrates. Multilayered structures comprising high- and low-refractive-index metal oxide, metal oxide hydrate, metal or metal fluoride layers may also be present, preferably with high- and low-refractive-index layers alternating.

Particularly suitable materials of high refractive index are, for example, TiO₂, ZrO₂, ZnO, SnO₂ and/or mixtures thereof. TiO₂ is particularly preferred. The thickness of these layers is in each case about 3 to 300 nm and preferably 20 to 200 nm.

Particularly suitable materials of low refractive index are, for example, SiO₂, SiO(OH)₂, Al₂O₃, AlO(OH), B₂O₃, MgF₂ and/or mixtures thereof. SiO₂ is particularly preferred. The thickness of the individual layers of these materials is between 3 and 300 nm, they are preferably thicker than 20 nm and up to 200 nm in thickness.

Overall, the materials of the additional layers should be selected and their layer thicknesses set depending on the layer material in such a way that the semitransparency of the interference pigments A and B is retained.

In the interference pigments A, the said substrates are provided with an iron-containing coating comprising metallic iron, which acts as outer, optically active layer. The layer thickness of the iron-containing coating is 1 to 300 nm, preferably 1 to 100 nm. The proportion of metallic iron in the iron-containing coating is 10 to 100% by weight, preferably 30 to 99% by weight and in particular 60 to 85% by weight, based on the iron-containing coating. The metallic iron is very particularly preferably present in the iron-containing coating in combination with FeO and/or Fe₃O₄. In addition, further simple or complex metal oxides, for example TiO₂, ilmenite or pseudobrookite, may also be present in the iron-containing coating. Pigments of this type are obtainable by reaction of a substrate coated with an iron oxide-containing coating in a reducing gas mixture comprising nitrogen and hydrogen with formation of metallic iron. This enables the provision of the iron-containing coating comprising metallic iron.

In the interference pigments B, the said substrates are provided with a coating comprising metallic tin and in addition at least one metal oxide, which coating acts as outer, optically active layer. The layer thickness of the coating comprising metallic tin and in addition at least one metal oxide is 1 to 300 nm, preferably 1 to 100 nm. The proportion of metallic tin in the coating is 0.01 to 50% by weight, preferably 0.05 to 20% by weight and in particular 0.1 to 10% by weight, based on the coating. Suitable as additional at least one metal oxide are tin oxide, titanium oxide, zirconium oxide and zinc oxide.

The metallic tin is very particularly preferably present in the coating in combination with titanium oxide as additional metal oxide. In addition, further simple or complex metal oxides, for example ilmenite or pseudobrookite, may also be present in the tin-containing coating.

Pigments of this type are obtainable by reaction of a substrate coated with a coating containing tin dioxide and optionally in addition at least one further metal oxide in a reducing gas mixture comprising nitrogen and hydrogen with formation of metallic tin. This enables the provision of the coating comprising metallic tin and in addition at least one metal oxide.

The reducing gas mixture comprising nitrogen and hydrogen to be employed for the preparation of the interference pigments A and B has a proportion of hydrogen in the range from 2.5 to 25% by vol., in particular from 4 to 10% by vol. and very particularly preferably from 5 to 8% by vol.

The reduction of the iron oxide-containing coating or the coating containing tin dioxide and optionally in addition at least one further metal oxide is carried out at temperatures from 400 to 1200° C., preferably from 500 to 1000° C. and particularly preferably from 550 to 900° C. The calcination duration is 15-240 minutes, preferably 30-120 minutes and in particular 30-90 minutes.

In a further embodiment of the present invention, the interference pigments A and B may furthermore be provided with an additional organic coating as the outer layer. Examples of coatings of this type are found, for example, in EP 0 632 109, U.S. Pat. No. 5,759,255, DE 43 17 019, DE 39 29 423, DE 32 35 017, EP 0 492 223, EP 0 342 533, EP 0 268 918, EP 0 141 174, EP 0 764 191, WO 98/13426 or EP 0 465 805, the disclosure content of which is hereby incorporated by way of reference.

Besides the improved optical properties already mentioned, interference pigments containing this organic coating, for example comprising organosilanes or organotitanates or organozirconates, additionally exhibit increased stability to weathering influences, such as, for example, humidity and light, which is of particular interest for industrial coatings and in the automobile sector. The process step of application of the organic coating can be carried out directly after the steps for preparation of the pigments or independently thereof. The substances applied here only comprise a proportion by weight of 0.1 to 5% by weight, preferably 0.5 to 3% by weight, of the pigment as a whole.

The pigment mixture according to the invention is prepared by mixing the interference pigments A and B, where the mixing can be carried out in all manners known to the person skilled in the art, for example by simple stirring into the application system. Complex grinding and dispersal of the pigments is not necessary.

Owing to their advantageous properties, the pigment mixtures according to the invention are suitable for a wide range of applications. The invention therefore also relates to the use of the pigment mixtures according to the invention in cosmetics, surface coatings, inks, plastics, films, in security printing, in security features in documents and identity papers, for colouring seed, for colouring foods, for laser marking or in medicament coatings and for the preparation of pigment compositions and dry preparations.

In the case of cosmetics, the pigment mixtures according to the invention are particularly suitable for products of decorative cosmetics, such as, for example, nail varnishes, colouring powders, lipsticks or eye shadows. The pigment mixtures according to the invention can of course also be combined in the formulations with cosmetic raw materials and assistants of any type. These include, inter alia, oils, fats, waxes, film formers, preservatives and assistants which generally determine the applicational properties, such as, for example, thickeners and rheological additives, such as, for example, bentonites, hectorites, silicon dioxide, Ca silicates, gelatine, high-molecular-weight carbohydrates and/or surface-active assistants, etc. The formulations comprising pigment mixtures according to the invention can belong to the lipophilic, hydrophilic or hydrophobic type. In heterogeneous formulations having discrete aqueous and non-aqueous phases, the particles according to the invention may be present in each case in only one of the two phases or also distributed over both phases.

The pH values of the aqueous formulations can be between 1 and 14, preferably between 2 and 11 and particularly preferably between 5 and 8. The concentrations of the pigment mixtures according to the invention in the formulation are not limited. They can be—depending on the application—between 0.001 (rinse-off products, for example shower gels) and 99% (for example gloss-effect articles for particular applications). The pigment mixtures according to the invention may furthermore also be combined with cosmetic active compounds. Suitable active compounds are, for example, insect repellents, UV A/BC protection filters (for example OMC, B3, MBC), anti-ageing active compounds, vitamins and derivatives thereof (for example vitamin A, C, E, etc.), self-tanning agents (for example DHA, erythrulose, inter alia) and further cosmetic active compounds, such as, for example, bisabolol, LPO, ectoin, emblica, allantoin, bioflavonoids and derivatives thereof.

In the case of use of the pigment mixtures in surface coatings and inks, all areas of application known to the person skilled in the art are possible, such as, for example, powder coatings, automobile paints, printing inks for gravure, offset, screen or flexographic printing and surface coatings in outdoor applications. The surface coatings and inks here may be, for example, radiation-curing, physically drying or chemically curing. For the preparation of printing inks or liquid coatings, a multiplicity of binders, for example based on acrylates, methacrylates, polyesters, polyurethanes, nitrocellulose, ethylcellulose, polyamide, polyvinyl butyrate, phenolic resins, maleic resins, starch or polyvinyl alcohol, amino resins, alkyd resins, epoxy resins, polytetrafluoroethylene, polyvinylidene fluorides, polyvinyl chloride or mixtures thereof, in particular water-soluble grades, is suitable. The surface coatings can be powder coatings or water- or solvent-based coatings, where the choice of the coating constituents is subject to the general knowledge of the person skilled in the art. Common polymeric binders for powder coatings are, for example, polyesters, epoxides, polyurethanes, acrylates or mixtures thereof.

The pigment mixtures according to the invention can in principle also be employed in the formulations in combination with other colorants or fillers of any type.

In addition, the pigment mixtures according to the invention can be used for pigmenting films and plastics, thus, for example, for agricultural sheeting, gift foils, plastic containers and mouldings for all applications known to the person skilled in the art. Suitable plastics for incorporation of the pigment mixtures according to the invention are all common plastics, for example thermosets or thermoplastics. The description of the potential applications and the plastics, processing methods and additives which can be employed is given, for example, in RD 472005 or in R. Glausch, M. Kieser, R. Maisch, G. Pfaff, J. Weitzel, Perlglanzpigmente [Pearlescent Pigments], Curt R. Vincentz Verlag, 1996, 83 ff., the disclosure content of which is hereby incorporated.

In addition, the pigment mixtures according to the invention are also suitable for use in security printing and in security-relevant features for, for example, counterfeiting-proof cards and identity papers, such as, for example, entry tickets, personnel identity cards, bank notes, cheques and cheque cards, and for other counterfeiting-proof documents. In the agricultural sector, the pigment mixtures can be used for colouring seed and other starting materials, in addition in the foods sector for pigmenting foods. The pigment mixtures according to the invention can likewise be employed for pigmenting coatings in medicaments, such as, for example, tablets or dragees.

All known thermoplastics, as described, for example, in Ullmann, Vol. 15, pp. 457 ff., Verlag VCH, can be used for laser marking using the mixture according to the invention. Suitable plastics are, for example, polyethylene, polypropylene, polyamides, polyesters, polyester-esters, polyether-esters, polyphenylene ether, polyacetal, polybutylene terephthalate, polymethyl acrylate, polyvinyl acetate, polystyrene, acrylonitrile-butadiene-styrene, acrylonitrile-styrene-acrylate, polycarbonate, polyether sulfones, polyether ketones and copolymers and/or mixtures thereof.

The mixture according to the invention is incorporated into the thermoplastic by mixing the plastic granules with the mixture and then shaping under the action of heat. During incorporation of the mixture, adhesives known to the person skilled in the art, organic polymer-compatible solvents, stabilisers and/or surfactants which are temperature-stable under the working conditions can be added to the plastic granules. The pigmented plastic granules are generally produced by initially introducing the plastic granules into a suitable mixer, wetting them with any additives and then adding and mixing in the mixture according to the invention. The mixture obtained in this way can then be processed directly in an extruder or injection-moulding machine. Marking using suitable radiation is carried out subsequently.

During the marking, use is preferably made of high-energy radiation, in general in the wavelength range from 157 to 10,600 nm, in particular in the range from 300 to 10,600 nm. Mention may be made here by way of example of CO₂ lasers (10,600 nm), Nd:YAG lasers (1064 or 532 nm) or pulsed UV lasers (excimer lasers). The excimer lasers have the following wavelengths: F₂ excimer laser (157 nm), ArF excimer laser (193 nm), KrCl excimer laser (222 nm), KrF excimer laser (248 nm), XeCl excimer laser (308 nm), XeF excimer laser (351 nm), frequency-multiplied Nd:YAG lasers having wavelengths of 355 nm (frequency-tripled) or 265 nm (frequency-quadrupled). Particular preference is given to the use of Nd:YAG lasers (1064 or 532 nm) and CO₂ lasers. The energy densities of the lasers employed are generally in the range from 0.3 mJ/cm² to 50 J/cm², preferably 0.3 mJ/cm² to 10 J/cm².

The laser inscription is carried out by bringing the specimen into the ray path of a pulsed laser, preferably a CO₂ or Nd:YAG laser. Furthermore, inscription using an excimer laser, for example via a mask technique, is possible. However, the desired results can also be achieved using other conventional types of laser which have a wavelength in a region of high absorption of the laser light-absorbent substance used. The marking obtained is determined by the irradiation time (or number of pulses in the case of pulsed lasers) and irradiation power of the laser and of the plastic system or surface-coating system used. The power of the lasers used depends on the particular application and can readily be determined by the person skilled in the art in each individual case.

On use of pulsed lasers, the pulse frequency is generally in the range from 1 to 30 kHz. Corresponding lasers which can be employed in the process according to the invention are commercially available.

The mixture according to the invention can be used for laser marking in all above-mentioned plastics. The plastics pigmented in this way can be used as mouldings in the electrical, electronics and motor vehicle industries. A further important area of application for laser inscription is in identity cards and plastic marks for the individual tagging of animals. The proportion of interference pigments in the plastic is 0.01 to 10% by weight, preferably 0.05 to 5% by weight and in particular 0.1 to 3% by weight in the case of laser marking in the applications. The labelling and inscription of casings, lines, key caps, ornamental strips and functional parts in the heating, ventilation and cooling sectors or switches, plugs, levers and handles which consist of the plastics pigmented with the pigments according to the invention can be carried out with the aid of laser light even in places which are difficult to access. The markings are distinguished by the fact that they are wipe- and scratch-resistant, are stable during subsequent sterilisation processes and can be applied in a hygienically clean manner during the marking process.

In the above-mentioned areas of application, the pigment mixtures according to the invention are likewise suitable for use in blends with all known organic or inorganic dyes and/or pigments. Organic pigments and dyes are, for example, monoazo pigments, disazo pigments, polycyclic pigments, cationic, anionic or nonionic dyes. Inorganic dyes and pigments are, for example, white pigments, coloured pigments, black pigments or effect pigments. Examples of suitable effect pigments are metal-effect pigments, pearlescent pigments or interference pigments, which are generally based on mono- or multicoated flakes based on mica, glass, Al₂O₃, Fe₂O₃, SiO₂, etc. Examples of structures and particular properties of the said pigments are given, for example, in RD 471001 or RD 472005, the disclosure content of which is hereby incorporated into the present invention by way of reference. In addition, further colorants which are suitable for blending with the mixtures according to the invention are luminescent dyes and/or pigments and holographic pigments or LCPs (liquid crystal polymers). The pigment mixtures according to the invention can be mixed with commercially available pigments and fillers in any ratio.

Fillers which may be mentioned are, for example, natural and synthetic mica, nylon powder, pure or filled melamine resins, talc, glasses, kaolin, oxides or hydroxides of aluminium, magnesium, calcium, zinc, BiOCl, barium sulfate, calcium sulfate, calcium carbonate, magnesium carbonate, carbon, and physical or chemical combinations of these substances. There are no restrictions regarding the particle shape of the filler. It can be, for example, flake-form, spherical or needle-shaped in accordance with requirements.

The pigment mixtures according to the invention are furthermore suitable for the preparation of flowable pigment compositions and dry preparations comprising one or more particles according to the invention, binders and optionally one or more additives. Dry preparations are also taken to mean preparations which comprise 0 to 8% by weight, preferably 2 to 8% by weight, in particular 3 to 6% by weight, of water and/or a solvent or solvent mixture. The dry preparations are preferably in the form of pellets, granules, chips, sausages or briquettes and have particle sizes of 0.2-80 mm. The dry preparations are used, in particular, in the preparation of printing inks and in cosmetic formulations.

The present invention likewise relates to cosmetics, surface coatings, inks, plastics, films, documents and identity papers, seed, foods or medicament coatings, pigment compositions and dry preparations comprising the pigment mixtures according to the invention.

The following examples are intended to explain the invention in greater detail, but without limiting it.

### EXAMPLES

a) Preparation of Interference Pigment A

100 g of mica having a particle size of 10-60 μm are heated to 75° C. with stirring in 1.9 l of demineralised water.

The pH of the suspension is adjusted to 3.0 using 10% hydrochloric acid. 200 g of a 30% FeCl₃ solution are then metered in, during which the pH is kept constant by simultaneous dropwise addition of 32% sodium hydroxide solution. The product is filtered off, washed, dried and reduced at 700° C. in a gas mixture comprising nitrogen and hydrogen (proportion of hydrogen: 8% by vol.), giving a lustrous pigment having silver interference, a grey-black mass tone and high gloss, whose coating consists of 74% by weight of metallic iron.

b) Preparation of Interference Pigment B

100 g of mica having a particle size of 10-60 μm are heated to 75° C. with stirring in 1.9 l of demineralised water.

The pH of the suspension is adjusted to 1.8 using 5% hydrochloric acid. This is followed by metered addition of a tin tetrachloride solution (comprising 3 g of SnCl₄.5 H₂O and 10 ml of concentrated hydrochloric acid in 90 ml of demineralised water), during which the pH is kept constant by simultaneous dropwise addition of 32% sodium hydroxide solution. A 30% titanium tetrachloride solution (180 g of TiCl₄ solution w=60%, dissolved in 180 g of demineralised water) is then added, during which the pH is kept constant by simultaneous dropwise addition of 32% sodium hydroxide solution. The product is filtered off, washed, dried and reduced at 850° C. in a gas mixture comprising nitrogen and hydrogen (proportion of hydrogen: 8% by vol.), giving a pigment comprising metallic tin having silver interference, a colour-neutral grey mass tone and high gloss.

c) Preparation of a Pigment Mixture

The pigments from Examples a) and b) are mixed in the ratio by weight of 1:0.75 (interference pigment A:interference pigment B). The mixture obtained in this way is incorporated into an acrylate-melamine surface coating in a concentration of 3.5% by weight and applied to a red-painted metal sheet, giving a glossy, silver-coloured coating which exhibits a slightly reddish colour impression on tilting.

### Comparative Example

The pigment from Example b) is mixed with carbon black in the ratio by weight of 1:0.06 (interference pigment B:carbon black). The mixture obtained in this way is incorporated into an acrylate-melamine surface coating in a concentration of 2.5% by weight and applied to a red-painted metal sheet, giving a glossy, silver-coloured coating in which the red substrate is no longer visible even when viewed at a flat angle.

Use Example Laser Marking:

PP granules (PP-HD, Stamylan PPH 10 from DSM) are processed by injection moulding by addition of 0.1% by weight of the mixture from Example c). The moulding obtained (platelet) is subsequently inscribed using an SHT Nd:YAG laser. At a pulse frequency of 2.5 kHz and a writing speed of 300 mm/s, the platelets exhibit a black, high-contrast and abrasion-resistant inscription. With increasing energy density, the inscription becomes increasingly dark.

1. Mixture of interference pigments, characterised in that the mixture comprises at least one interference pigment A comprising a substrate and a coating comprising metallic iron, and an interference pigment B comprising a substrate and a coating comprising metallic tin and in addition at least one metal oxide.

2. Mixture according to claim 1, characterised in that the proportion of metallic iron in the iron-containing coating is 10 to 100% by weight, based on the iron-containing coating.

3. Mixture according to claim 1, characterised in that the metallic iron is present in the iron-containing coating in combination with FeO and/or Fe₃O₄.

4. Mixture according to claim 1, characterised in that the proportion of metallic tin in the coating comprising metallic tin is 0.01 to 50% by weight, based on the coating comprising metallic tin and in addition at least one metal oxide.

5. Mixture according to claim 1, characterised in that the additional metal oxide is titanium oxide.

6. Mixture according to claim 1, characterised in that the substrates of interference pigments A and B are different from one another and comprise synthetic or natural mica, phyllosilicates, glass, SiO₂, Al₂O₃, TiO₂, graphite and/or BiOCl.

7. Mixture according to claim 1, characterised in that one or more layers comprising metal oxides, metal oxide hydrates, metal suboxides, metals, metal fluorides, metal nitrides, metal oxynitrides and/or mixtures thereof are additionally present between the coating comprising metallic iron and the substrate in interference pigment A and/or between the coating comprising metallic tin and in addition at least one metal oxide and the substrate in interference pigment B.

8. Pigment mixture according to claim 1, characterised in that the ratio of interference pigment A to interference pigment B is 99:1 to 1:99.

9. Pigment mixture according to claim 1, characterised in that the interference pigments A and B are furthermore provided with an additional organic coating as the outer layer.

10. A method of making mixtures in cosmetics, surface coatings, inks, plastics, films, in security printing, in security features in documents and identity papers, for colouring seed, for colouring foods, for laser marking or in medicament coatings and for the preparation of pigment compositions and dry preparations comprising incorporating a mixture of claim 1.

11. Method according to claim 10, characterised in that the mixtures are in the form of a blend with organic or inorganic dyes and/or pigments.

12. Cosmetics, surface coatings, inks, plastics, films, documents and identity papers, seed, foods or medicament coatings, pigment compositions and dry preparations comprising mixtures according to claim 1.
